(19) United States
(12) Patent Application Publication
(10) Pub. No.: US 2021/0298821 A1
(43) Pub. Date: Sep. 30, 2021

(54) ENDOSCOPIC VESSEL HARVESTER WITH INTEGRATED SUCTION LINE

(71) Applicant: TERUMO CARDIOVASCULAR SYSTEMS CORPORATION, Ann Arbor, MI (US)

(72) Inventor: Safi Siddiqui, Chestertown, MD (US)

(21) Appl. No.: 16/835,417

(22) Filed: Mar. 31, 2020

(51) Int. Cl. A61B 18/14; A61B 1/00; A61B 1/015

(52) U.S. Cl. CPC A61B 18/1482; A61B 2018/00595; A61B 1/015; A61B 1/00094

(57) ABSTRACT

An endoscopic vessel harvester includes a vessel keeper with a capture frame capturing a target vessel within a tunnel that has been dissected along the target vessel. A cutter member cuts and cauterizes side branches while the target vessel is slidably captured in the vessel keeper. A fixed supporting rod and a movable side rod engage the frame. The movable side rod has an opened position to admit the target vessel and has a closed position to slidably capture the target vessel. One of the fixed support rod or the movable side rod is comprised of a hollow cylinder open at the distal end and at the proximal end, and wherein the distal end of the hollow cylinder fluidically communicates with the tunnel through the capture frame. A suction tube connects to the proximal end of the hollow cylinder, allowing pooled fluid around the capture frame to be removed.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to endoscopic harvesting of blood vessels, and, more specifically, to providing suction capability in a harvesting tool for endoscopically removing fluids during subcutaneous dissection and cauterizing of a target vessel.

In coronary artery bypass grafting (CABG), a blood vessel or vessel section, such as an artery or vein, is “harvested” (i.e., removed) from its natural location in a patient's body for use as a graft. After removal, the section of blood vessel is joined between an arterial blood source and the coronary artery that is to be bypassed. Among the preferred sources for the vessel to be used as the bypass graft are the saphenous vein in the legs and the radial artery in the arms.

Endoscopic surgical procedures for harvesting a section of a vessel (e.g., the saphenous vein) subcutaneously have been developed in order to avoid disadvantages and potential complications of older harvesting techniques wherein a continuous incision (e.g., along the leg) was made for the full length of the desired vessel section in order to provide adequate exposure for visualizing the vessel and for introducing surgical instruments to sever, cauterize, and ligate the tissue and side branches of the vessel. One such minimally-invasive technique employs a small incision for locating the desired vessel and for introducing one or more endoscopic harvesting devices. Primary dissection occurs by introduction of a blunt dissecting instrument through the incision to create a working space and to separate the vessel from the surrounding tissue. Then a cutting instrument is introduced into the working space to sever the blood vessel from the connective tissue surrounding the section to be harvested and any side branches of the blood vessel. The branches may be clipped and/or cauterized.

In one typical procedure, the endoscopic entry site is located near the midpoint of the vessel being harvested, with dissection and cutting of branches proceeding in both directions along the vessel from the entry site. In order to remove the desired section of the blood vessel, a second small incision, or stab wound, is made at one end thereof and the blood vessel section is ligated. A third small incision is made at the other end of the blood vessel section which is then ligated, thereby allowing the desired vessel section to be completely removed through the first incision. Alternatively, only the first two incisions may be necessary if the length of the endoscopic device is sufficient to obtain the desired length of the blood vessel while working in only one direction along the vessel from the entry point.

An example of a commercially available product for performing the endoscopic vessel harvesting described above is the VirtuoSaph™ Endoscopic Vein Harvesting System from Terumo Cardiovascular Systems Corporation of Ann Arbor, Mich. Endoscopic vessel harvesting systems are described in U.S. Pat. No. 8,465,488 to Maeda et al, U.S. Pat. No. 8,702,700 to Maeda et al, and U.S. Pat. No. 7,547,314 to Kadykowski, which are each incorporated herein by reference in their entirety. After harvesting, the vessel is inspected and prepared for surgery by checking for leaks or other defects. The prepared vessel is then stored in a preservative fluid until needed.

In the VirtuoSaph™ System, the cutting tool for severing and cauterizing branches includes a V-cutter extendable from the distal end of the tool, wherein a V-shaped tip of the V-cutter guides a branch to be cut into a longitudinal slit. Electrodes adjacent the slit are electrically energized with a high frequency voltage in order to cauterize and sever the branch by coagulation.

A lockable vessel or vein keeper (V-keeper) is independently controllable (e.g., longitudinally) alongside the V-cutter at the distal end of the cutting tool in order to i) selectably hold the target vessel in a desired position for aligning the V-cutter with the side branches or connective tissue attached to the vessel, and ii) guide the cutting tool along the dissected vessel. The vein keeper may include a vein keeper shaft extending from a proximal handle of the tool to the distal tip where it is fixed to one lateral side of a substantially C-shaped capture frame or base. The vein keeper shaft is extendable/retractable in the longitudinal direction of the tool in order to locate the base according to a desired distal extension. A lock shaft is slidable within the frame to selectably close the open side of the C-shape of the frame. The lock shaft likewise extends into the handle where it connects to a mechanism to be manipulated forward and backward by a user to 1) open the side of the frame for moving a target vessel into or out of the center space within the frame, or 2) enclosing the center space to lock the target vessel within the frame.

For viewing the internal anatomy, the V-cutter, and the V-keeper during a vessel harvesting procedure, an endoscope is longitudinally inserted into the tool to place the endoscopic lens near the tip. During harvesting, unintended bleeding can occur in the dissected tunnel (e.g., from inadvertent damage to the vessel). Blood and other fluids in the tunnel can quickly pool, obstructing the view from the endoscope and complicating the procedure. It would be undesirable and disruptive to remove the cutting tool in order to access the tunnel with a suction tube to clear the pooled fluids. In addition, it would be difficult to run a suction line as an additional component within the cutting tool due to a shortage of available space within the longitudinal body of the tool.

### SUMMARY OF THE INVENTION

In one aspect of the invention, an endoscopic vessel harvester comprises a longitudinal insertion member having a proximal end with a handle and a distal end adapted for insertion into a tunnel dissected along a target vessel within a body of a patient. A vessel keeper is extendably mounted at the distal end of the insertion member comprising a capture frame with a fixed supporting rod and a movable side rod. The movable side rod has an opened position to admit the target vessel and has a closed position to slidably capture the target vessel. Each rod has a distal end engaging the capture frame and a proximal end in the handle. One of the fixed support rod or the movable side rod is comprised of a hollow cylinder open at the distal end and at the proximal end. The distal end of the hollow cylinder fluidically communicates with the tunnel. A cutter member is extendably mounted at the distal end of the insertion member having a cauterizing element adapted to contact side branches of the target vessel and to cut and cauterize the side branches while the target vessel is slidably captured in the vessel keeper. A handle mechanism is coupled to the proximal ends of the rods to manually adjust longitudinal positions of the rods independently. A suction tube with a first end is coupled to the proximal end of the hollow cylinder, and a second end outside the handle is configured to attach to a suction supply.

### BRIEF DESCRIPTION OF THE DRAWINGS

FIG. 1 is a side view of a vessel harvester of the invention.

FIG. 2 is an endoscopic view of the harvester inside the body of a patient during harvesting of a vessel.

FIG. 3 represents the endoscopic view of FIG. 2 wherein a pooled liquid such as blood has pooled to obscure portions of the view.

FIG. 4 is a perspective view of the distal end of the harvester.

FIG. 5 is a top view of the distal end of the harvester.

FIG. 6 is a perspective view of the capture frame in partial cross section.

FIG. 7 is a perspective view of the capture frame and the hollow supporting rod in cross section.

FIG. 8 is a longitudinal cross section of a harvester of the present invention.

FIG. 9 is a perspective view of a capture frame according to another embodiment.

FIG. 10 is a cross-sectional view of the capture frame of FIG. 9.

### DETAILED DESCRIPTION OF PREFERRED EMBODIMENTS

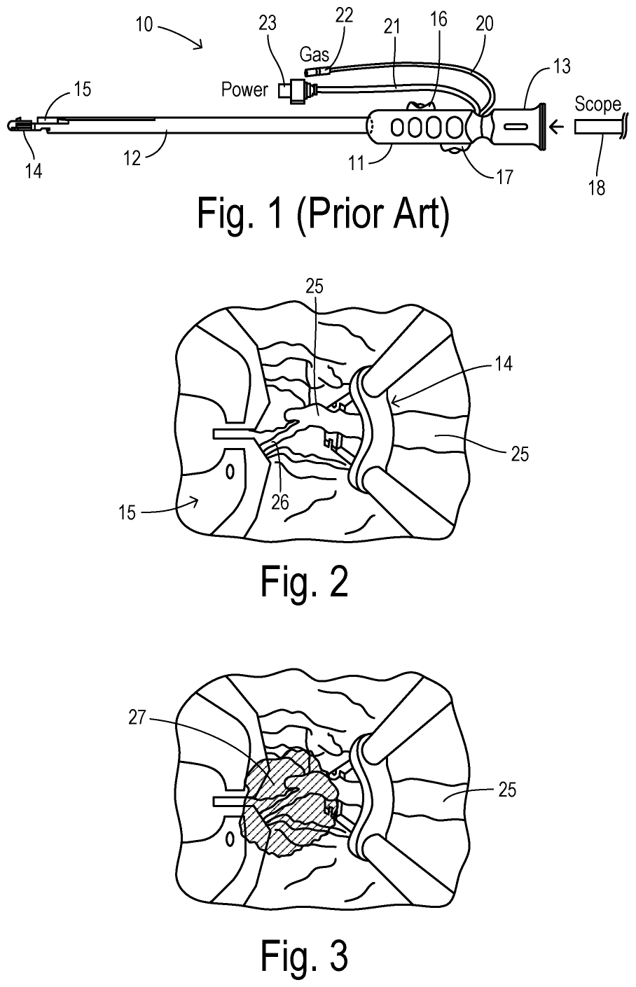

FIG. 1 shows a known type of harvester tool 10 used to grasp the target vessel being dissected and to sever any branches or connective tissue connecting to the vessel. Harvester 10 is inserted into a working tunnel along a target vessel that is created using a dissector rod (not shown). Harvester 10 has a handle 11 connected to an elongated sleeve member or insertion member 12 and to an endoscope receiver 13. An endoscope 18 is longitudinally inserted into receiver 13 so that a lens at the end of endoscope 18 captures a view at the distal top of harvester 10. At the distal end of insertion member 12 are a vessel-keeper (V-keeper) 14 which is a capture frame for retaining the vessel being dissected and a V-cutter 15 for severing side branches and connective tissue. V-keeper 14 is manipulated by V-keeper buttons 16 on handle 11. V-cutter 15 is extended or retracted by manipulating a V-cutter extender button 17 on handle 11. An insufflator tube 20 can be connected to a source of gas such as CO₂ by a connector 22 to deliver insufflation gas to the distal end of insert member 12. A bipolar cord 21 has a connector 232 at one end for connecting to a source of high frequency voltage, and includes conductors for supplying the voltage to electrodes on V-cutter 15.

FIG. 2 is an endoscopic view as seen during vessel harvesting wherein a target vessel (e.g., saphenous vein) 25 is retained within an opening of V-keeper 14 within a tunnel around vessel 25 created previously during blunt dissection. V-cutter 15 is in position for extending toward a side branch 26 for cauterizing and severing it to prepare a section of vessel 25 for removal. FIG. 3 shows the same endoscopic view after accumulation of blood and/or other fluids into a pool 27 that obscures side branch 26 and a portion of vessel 25.

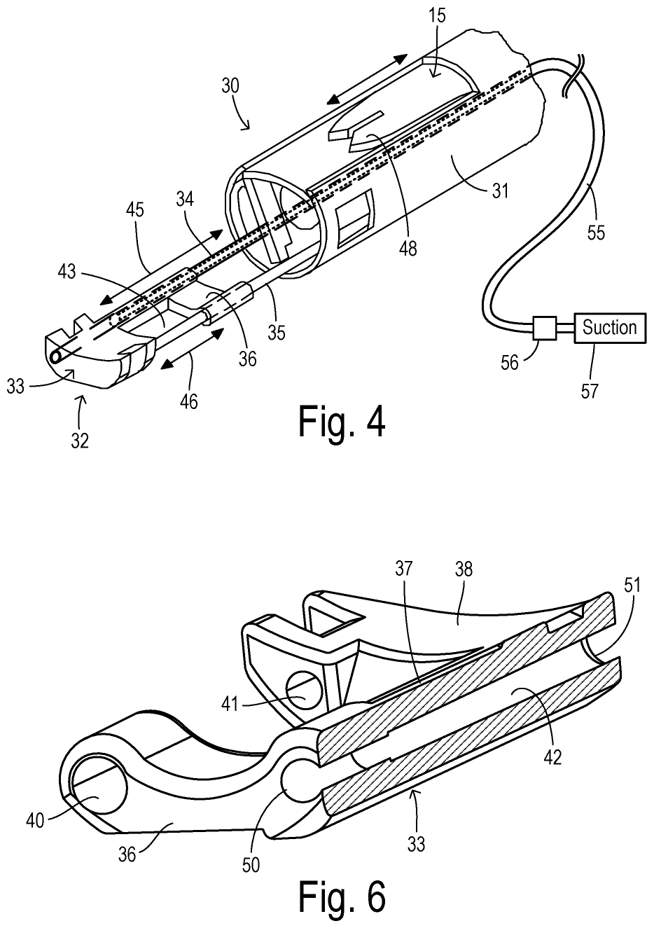

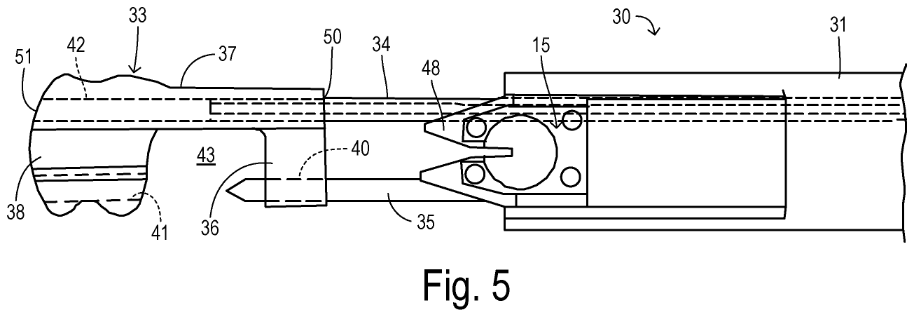

In order to integrate a suctioning capability into a harvester tool, one of the solid rods formerly uses to support the capture frame or to provide locking/unlocking of the capture area of the frame is replaced with a hollow, cylindrical rod. FIGS. 4 and 5 shows a portion of a harvester tool 30 integrally incorporating a suction line. A main sheath or rod 31 extends from a handle (not shown) to the distal end of tool 30 where a V-keeper 32 includes a guide/capture frame 33 mounted to a fixed support rod 34 and a movable rod 35. Capture frame 33 has a first leg 36, a second leg 37, and a third leg 38. First leg 36 has a bore or passage 40 for slidably receiving movable rod 35. Third leg 38 has a bore 41 axially aligned with bore 40. Second leg 37 has a bore 42 parallel with bores 40 and 41, and aligned to receive fixed support rod 34. Support rod 34 is fixed with respect to frame 32. Support rod 34 and frame 33 are movable together longitudinally (shown by arrow 45 in FIG. 4) by manipulating a control mechanism in the handle via buttons on the handle (e.g., buttons 16 and 17 in FIG. 1).

The C-shaped capture frame 33 (defined by legs 36-38) and movable rod 35 together define an internal opening 43. The vein or other vessel to be harvested is maneuvered into opening 43, and then the V-keeper buttons on the handle are manipulated to extend rod 35 along an arrow 46 at one side of capture frame 33 (from the position shown in FIG. 5 to the position shown in FIG. 4) in order to close opening 43 and thereby retain the vessel. V-cutter 15 includes a V-tip 48 with a central slit mounted to an extendable guide that is manipulated by the V-cutter button on the handle in order to place side branches into the slit.

As shown in FIGS. 4 and 5, bore or passageway 42 extends continuously through capture frame 33. A first end 50 of bore 42 receives support rod 34. Rod 34 extends part way into bore 42, and is rigidly affixed by a press fit, adhesive bonding, welding, or other means. A second end 51 of bore 42 at the distal end of capture frame 33 functions as a suction port. Support rod 34 is a hollow rod (e.g., cylinder) from end to end to provide fluid communication from the suction port at bore end 51 to a proximal end of rod 34 in the handle. Preferably, hollow rod 34 is formed of stainless steel or other rigid, biocompatible material. As shown in FIG. 4, a flexible tubing 55 which is connected at one end to the proximal end of rod 34 has a control valve 56 at the other end for connecting to a suction supply (e.g., a vacuum source as commonly available in surgical settings).

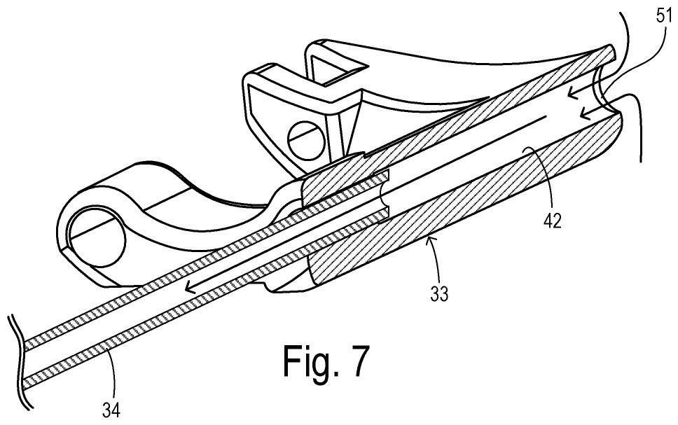

FIGS. 6 and 7 show capture frame 33 and support rod 34 in greater detail. With support rod 34 installed in bore 42 (e.g., by press fit), a continuous path through the interior of rod 34 and bore 42 creates fluid communication traversing the harvesting tool from suction port 51 to a suction source via the handle and a flexible tube exiting the handle.

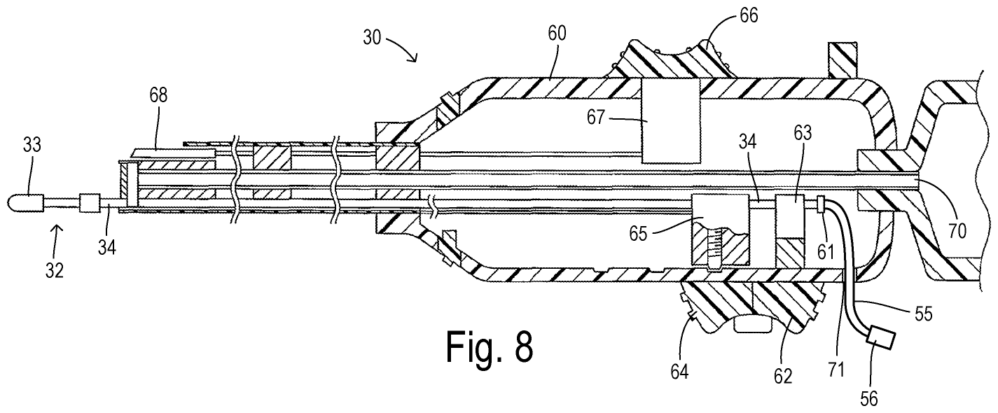

Referring to FIG. 8, harvesting tool 30 includes a handle 60 configured to control V-keeper 32 and a V-cutter 68. A connection 61 joins a proximal end of hollow support rod 34 to flexible tubing 55 for fluid communication to the distal end of capture frame 33. A first V-keeper button 62 is linked to rod 34 by a control mechanism 63, so that a user can manually extend or retract V-keeper 32 as a whole by a selected distance. A second V-keeper button 64 is linked to movable rod 35 by a control mechanism 65 for selectably opening and closing the opening 43. Similarly, a V-cutter button 66 and a control mechanism 67 are used to extend/retract V-cutter 68. A center lumen 70 is configured to convey the endoscope within harvesting tool 30.

Flexible tubing 55 exits from handle 60 via an aperture 71. Tubing 55 can be placed adjacent other flexible tubing exiting handle 60, such as tubing for insufflation gas. Tubing 55 can be comprised of a polymeric material.

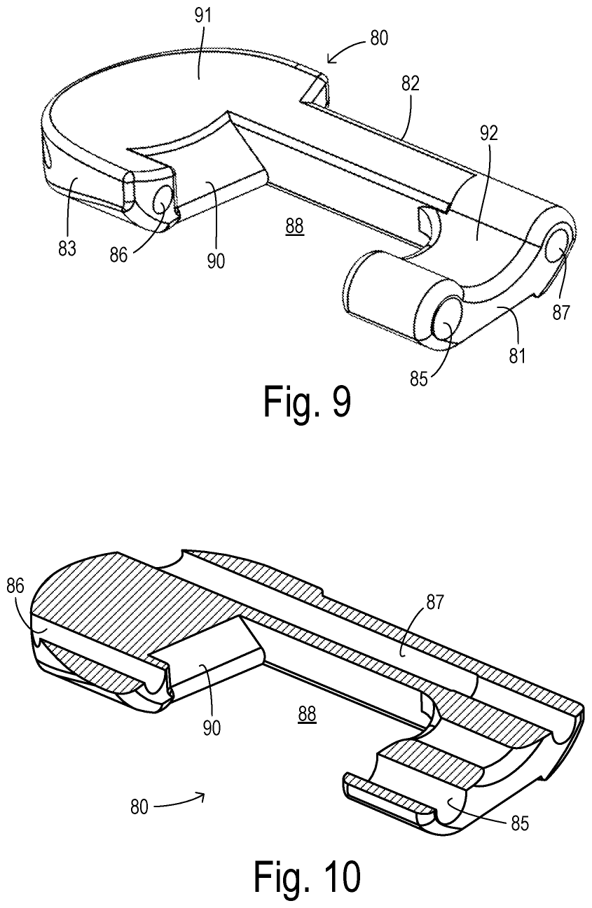

FIGS. 9 and 10 show another embodiment of a capture frame 80 with a first leg 81, a second leg 82, and a third leg 83. A passageway 85 extends through first leg 81 and is coaxial with a passageway 86 in third leg 83 for slidably receiving a movable rod. A passageway 87 extends through second leg 82 for fixedly receiving a support rod. Frame 80 is C-shaped with a center opening 88. A ramp surface 90 in third leg 83 adjacent opening 88 is providing for guiding a vessel that is locked within opening 88. Upper surfaces 91 and 92 of third leg 83 and first leg 81, respectively, provide curved profiles also adapted for guiding the vessel as the V-keeper slides along the vessel.

Although the hollow rod for providing a suction line across the harvesting tool has been shown as the fixed support rod attached to the V-keeper, the movable rod for locking/unlocking the center opening could alternatively be used to provide the suction line. Passageway 86 extends across third leg 83 so that if the movable rod is hollow to provide the suction line, then continuous fluid communication is not interrupted if the movable rod is in a fully extended position to lock the center opening.

What is claimed is:

1. An endoscopic vessel harvester comprising:
a longitudinal insertion member having a proximal end with a handle and a distal end adapted for insertion into a tunnel dissected along a target vessel within a body of a patient;
a vessel keeper extendably mounted at the distal end of the insertion member comprising a capture frame with a fixed supporting rod and a movable side rod, wherein the movable side rod has an opened position to admit the target vessel and has a closed position to slidably capture the target vessel, wherein each rod has a distal end engaging the capture frame and a proximal end in the handle, wherein one of the fixed support rod or the movable side rod is comprised of a hollow cylinder open at the distal end and at the proximal end, and wherein the distal end of the hollow cylinder fluidically communicates with the tunnel;
a cutter member extendably mounted at the distal end of the insertion member having a cauterizing element adapted to contact side branches of the target vessel and to cut and cauterize the side branches while the target vessel is slidably captured in the vessel keeper;
a handle mechanism coupled to the proximal ends of the rods to manually adjust longitudinal positions of the rods independently; and
a suction tube having a first end coupled to the proximal end of the hollow cylinder and having a second end outside the handle configured to attach to a suction supply.

2. The harvester of claim 1 wherein the fixed supporting rod is comprised of the hollow cylinder.

3. The harvester of claim 2 wherein the capture frame has an internal bore with a first end receiving the distal end of the fixed supporting rod, and wherein a predetermined length along the distal end of the fixed supporting rod is rigidly retained in the internal bore.

4. The harvester of claim 3 wherein the internal bore has a second end emerging from the capture frame to form a suction port fluidically communicating with the hollow cylinder.

5. The harvester of claim 4 wherein the internal bore extends longitudinally through the capture frame, wherein the second end emerges at a distal end of the capture frame.

6. The harvester of claim 1 wherein the fixed supporting rod and the movable side rod are comprised of stainless steel.

7. The harvester of claim 1 wherein the rod having the hollow cylinder extends within the handle such that the handle mechanism attaches to the rod between the proximal and distal ends of the rod.

8. The harvester of claim 1 wherein the suction tube is comprised of a flexible tubing.

9. The harvester of claim 1 further comprising:
a control valve attached to the second end of the tube for controlling fluid communication to the suction supply.
